(19) United States
(12) Patent Application Publication
(10) Pub. No.: US 2011/0002968 A1
(43) Pub. Date: Jan. 6, 2011

(54) COSMETIC OR DERMATOLOGICAL COMPOSITION CONTAINING AN ORCHID EXTRACT, AND COSMETIC CARE METHOD USING SAID COMPOSITION

(75) Inventors: Virginie Leplanquais, Fay-aux-Loges (FR); Nancy Sauvan, Orleans (FR); Virginie Pecher, La Chapelle Saint Mesmin (FR); François Gerard, Villars-sur-Doron (FR)

Correspondence Address: MERCHANT & GOULD PC, P.O. BOX 2903, MINNEAPOLIS, MN, 55402-0903, US

(73) Assignee: LVMH RECHERCHE, Saint Jean de Braye (FR)

(21) Appl. No.: 12/745,654

(22) PCT Filed: Nov. 28, 2008
(86) PCT No.: PCT/FR08/52154

§ 371 Date: Sep. 8, 2010

(30) Foreign Application Priority Data
Nov. 30, 2007 (FR) 0759482

(51) Int. Cl. A61K 36/898; A61K 8/97; A61K 8/02; A61K 9/00; A61P 17/00; A61Q 19/08

(52) U.S. Cl. 424/401; 424/725; 424/773; 424/779; 424/774; 424/484

(57) ABSTRACT

Cosmetic or dermatological compositions contain an orchid extract obtained from at least a part of the species Vanda teres.

These compositions and extracts are used as a cosmetic or dermatological agent for maintaining the structure of the skin, in particular by limiting the degradation of the extracellular matrix of the upper layers of the dermis and of the epidermis, and/or for reducing or delaying the effects of skin ageing, in particular the formation of wrinkles, and/or for obtaining a protective, corrective or restructuring effect on the skin, and/or as an anti-inflammatory agent.

A method of cosmetic care for the skin is particularly intended for obtaining an effect of prevention or slowing down of the appearance of skin ageing signs.

The invention relates to a cosmetic or dermatological composition containing an orchid extract and to methods of cosmetic care using said composition.

Orchids (Orchidaceae) are the subject of numerous research studies in the medicament or cosmetics field, aimed at identifying new compounds having advantageous properties.

Orchids of the Vanda genus are epiphytic or epilithic orchids of the tropical forests of Asia and of Australia.

They are monopodial orchids of low-altitude forests with a high atmospheric humidity.

The genus comprises about fifty species with many hybrids.

### PRIOR ART

To date, research on the plants of Vanda genus is based on the knowledge and uses of these plants by local populations.

Thus, Bulpitt (“The uses and misuses of orchids in medicine”, QJM, 2005, 98 (9), 625-31) indicates that the orchid Vanda coerulea appears to be part of the Indian Pharmacopeia, without, however, citing references.

The author indicates that, surprisingly, and despite a number of different alkaloids present in plant tissues, no medical use has been demonstrated to date.

Manandhar et al. (Fitoterapia, 1994, 65 (1), 7-13) have described the use of Vanda cristata in the Kaski district of Nepal, for treating cuts and wounds using a paste based on the plant.

Sigh et al. have studied the traditional phytotherapy with medicinal plants used by the Tharu tribe of the Nainital district in the Uttar Pradesh region in India (Int. J. Pharmacogn., 1994 32 1, 51-58). These authors have described the use of a paste based on Vanda tessallata (synonym: Vanda roxburghii) obtained from the whole plant, and applied with salt to an area of bone fracture.

The publication also discloses the use of an aqueous extract of this same plant, administered orally for the same purpose.

Skin care compositions containing an extract of the plant Vanda roxburghii have also been described in Japanese patent application JP 2006-257056, this extract being used in this composition as an oestrogenic agent for the treatment of skin ageing.

Chawla et al. (Ind. J. Pharm. Sci., 1992, 54 (4), 159-61) have described the anti-inflammatory properties of an organic solvent-based extract obtained from Vanda roxburghii roots.

These extracts are obtained, respectively, with ether, chloroform and methanol.

The authors have demonstrated the presence of an alkyl ferulate, more specifically acetyltetracosyl ferulate, in the ether extract, and of beta-silosterol-D-glucoside in the chloroform extract.

The anti-inflammatory activity is measured by means of the method described in the publication Indian J. Chem, Chawla et al., 1991, 30B, 773.

These extracts have an anti-inflammatory activity of varying strength and which is less than that of ibuprofen.

The orchid Vanda teres has been the subject of very little scientific publication. It will be noted, however, that, in the Indian Journal of Environment and Ecoplanning (2007), 13 (1-2), 91-100, it has been possible to use extracts of the plant Papilionanthe teres (synonym of Vanda teres) for treating debility, by applying them to the forehead of patients during high fevers, and that, moreover, it has been possible to use stem extracts of this same plant for protecting against colds and coughs.

It will also be noted that the publication entitled “Karyotype analysis of some sarcanthine orchids” in the American Journal of Botany, Volume 50, n° 1, pages 73-79, 1963, which relates to an analytical study of various orchid karyotypes, mentioned, inter alia, Vanda teres.

It has now been discovered by the inventors of the present invention that, completely unexpectedly, orchids of the species Vanda teres exhibit noteworthy cosmetic and dermatological activities, in particular noteworthy properties of prevention or slowing down of the appearance of skin ageing signs associated with noteworthy anti-inflammatory properties.

The applicant has thus shown, in particular, that an extract of Vanda teres exhibits properties that are particularly advantageous in the cosmetics field, in the case in point an activity such that said extract can be used to contribute to maintaining the integrity of the structure of the skin.

This is because this extract inhibits the MMP-2 and 9 metalloproteinase enzymes responsible, inter alia, for the degradation of the collagen fibers of the extracellular matrix, and increases the expression of the IL-10 gene.

These various activities result in a preventive and slowing down effect on the appearance of skin ageing signs.

In addition to these unexpected properties, the extract of the invention also exhibits an anti-inflammatory activity through inhibition of the secretion of inflammation mediators, in particular interleukin 8 (IL-8) and prostaglandin E2 (PGE₂).

The extract obtained from orchids of the species Vanda teres can thus be used as an active agent in a cosmetic composition comprising at least one cosmetically acceptable excipient, and intended to be applied to at least a part of the skin of the face or the body, for reducing or delaying the effects of skin ageing.

This extract can also be advantageously used in cosmetic or dermatological compositions for its noteworthy anti-inflammatory properties.

Purposes of the Invention

The principal purpose of the invention is to provide a novel cosmetic or dermatological agent capable of reducing or delaying the effects of skin ageing, by more particularly maintaining the integrity of the structure of the skin, and also of combating skin inflammations, and a cosmetic or dermatological composition comprising said active agent.

A purpose of the invention is also to solve the technical problem by means of a particularly simple, relatively inexpensive solution that can be used on the industrial and cosmetic scale.

### SUMMARY OF THE INVENTION

According to a first aspect, the invention relates to a cosmetic or dermatological composition containing at least one extract of an orchid of the Vanda genus, obtained from at least a part of an orchid of the species Vanda teres, in solution or in a dispersion in a cosmetically or dermatologically acceptable carrier compatible with topical application to the skin.

According to a second aspect, the invention relates to the use of this extract in a cosmetic or dermatological composition, as a cosmetic or dermatological agent for maintaining the structure of the skin, in particular by limiting the degradation of the extracellular matrix of the upper layers of the dermis and of the epidermis, and/or for reducing or delaying the effects of skin ageing, in particular the formation of wrinkles, and/or for obtaining a protective, corrective or restructuring effect on the skin, and/or as an anti-inflammatory agent.

According to a third aspect, the invention relates to a method of cosmetic care for the skin, intended in particular to obtain an effect of prevention or slowing down of the appearance of skin ageing signs, comprising the application, to at least a part of the body or of the face, of a cosmetic composition which is the subject of the first aspect of the invention.

### DETAILED DESCRIPTION OF THE INVENTION

The first subject of the present invention is therefore a cosmetic or dermatological composition containing an extract obtained from a plant material formed from orchids of the species Vanda teres.

The plant material used may be the whole plant or a part of the plant, such as the leaves, the stem, the flowers or the roots.

The extract is preferably obtained from the stems and/or the roots and/or the leaves of the orchid, preferably the stems.

The extract is prepared by various extraction methods known to those skilled in the art.

However, the extraction is preferably carried out by bringing the selected plant material into contact with a polar solvent or a mixture of polar solvents.

Prior to this step of bringing at least a part of the plant into contact with at least one polar solvent, this plant part may be optionally dried and/or ground.

As polar solvent or mixture of polar solvents that can be used for the extraction step, a solvent or a mixture of solvents chosen from water, a C1-C4 alcohol, for example ethanol, and a C2 to C6 glycol preferably chosen from glycerol, butylene glycol and propylene glycol, and mixtures thereof will advantageously be chosen.

According to one preferred embodiment of the invention, the extraction is carried out using an aqueous-alcoholic mixture, in particular, a mixture of water and ethanol, preferably a mixture of water and ethanol respectively in a ratio of about 90/10 v/v.

According to another variant of the invention, the extraction may also be carried out by a process using a polar solvent in subcritical state, said solvent being advantageously water in subcritical state.

Prior to the extraction step itself, the plant material may have been dried and/or ground.

According to one preferred embodiment of the extraction, the plant material is in the dry and ground state.

The extraction may also optionally comprise an additional step constituted of a treatment of the extract aimed at partially or completely decoloring it, or at purifying it.

This decoloring step may, for example, be constituted of a treatment of the extract with a solution of a polar solvent or of a mixture of polar solvents, preferably of a treatment with a solution of ethanol/water in a ratio of about 70/30 v/v, in the presence of active carbon particles.

The extraction may be completed with a step of partial or complete elimination of the extraction solvents.

In the first case, the extract is generally concentrated until an aqueous concentrate devoid of significant amounts of organic solvent is obtained; in the second case, a dry residue is obtained.

Alternatively, the product of the extraction step may be lyophilized or atomized so as to be in the form of a powder.

The powder may be used as it is in a cosmetic or dermatological composition according to the invention or may be redispersed in a solvent or a mixture of solvents.

In general, the product of the extraction step may be dissolved or dispersed in a solvent or a mixture of solvents, so as to be used as an active agent in the cosmetic or dermatological compositions of the invention.

The solvent or the mixture of solvents in which the extract is dissolved or dispersed may be identical to or different than that having served for the extraction.

The extract of the invention may also be adsorbed onto a support advantageously chosen from porous or nonporous nylon powders, and micas, or any lamellar mineral substance.

In this case, the extract used is preferably an aqueous extract.

The cosmetic or dermatological composition according to the invention comprises an effective amount of extract of the invention for obtaining the desired effect.

The composition according to the invention thus preferably comprises from 0.001% to 5%, preferably from 0.01% to 1%, by dry weight of extract of Vanda teres.

The tests carried out by the inventors have shown that the properties of the compositions of the invention linked to the presence of the extract of Vanda teres can also be obtained or improved in cosmetic or dermatological compositions in which the extract is combined with other active agents having cosmetic effects similar and/or complementary to the extract of the invention.

Thus, the extract of the invention, alone or in combination with other active agents that contribute to maintaining the integrity of the structure of the skin, may also advantageously be combined with one or more active agents chosen from UVA and/or UVB screens, free-radical scavenger substances, calmative substances, substances for lightening the complexion and/or for regulating pigmentary disorders of the skin, and substances having a moisturizing effect.

The inventors have also discovered that the noteworthy cosmetic or dermatological properties linked to the presence, in the composition, of an extract of at least a part of the Vanda teres plant can, in a good many cases, be further greatly improved when this composition also contains at least one extract of at least one other plant belonging to the orchid family (Orchidaceae).

This additional extract of another plant belonging to the orchid family may in particular be an extract of at least one orchid of the Vanda genus, and most particularly an extract of at least a part of an orchid of the species Vanda denisoniana and/or of at least a part of an orchid of the species Vanda coerulea.

The compositions of the invention may comprise, at the same time, an extract of at least a part of the plant Vanda teres combined with at least one extract of at least a part of another orchid of the Vanda genus, as defined above, and optionally at least one other extract of another plant belonging to the orchid family.

According to one particularly advantageous variant, the compositions of the invention comprise at least one Vanda teres stem extract and/or one Vanda teres root extract, in combination with a Vanda denisoniana stem extract and/or a Vanda coerulea stem extract.

The tests carried out by the inventors have in fact shown that the presence, in the same composition, of at least one Vanda teres extract combined with at least one extract of another orchid of the Vanda genus results, in many cases, in improved cosmetic or dermatological properties.

The inventors have even, in certain cases, been able to demonstrate real synergies observed by mixing at least one Vanda teres extract with at least one other extract of at least a part of at least one other orchid of the Vanda genus.

Thus, the tests carried out by the inventors have been able to make it possible to demonstrate that the extracts of Vanda teres roots, even if these parts are not in themselves the most active parts of the plant, considerably improve the properties of prevention or of slowing down of the appearance of skin ageing signs or the anti-inflammatory properties observed with extracts of a plant of the Vanda genus.

As emerges from the examples hereinafter, by way of example of combinations which cause a significant improvement or even which produce a real synergy, mention will be made of the combinations of Vanda teres stem extracts with Vanda denisoniana stem and/or root extracts and/or with Vanda coerulea stem extracts.

It will in particular be noted that it has been possible to observe real synergy effects, both with regard to the activity of prevention or slowing down of the appearance of skin ageing signs and with regard to the anti-inflammatory activity, by mixing, in the composition, a Vanda teres stem extract, a Vanda teres root extract, a Vanda denisoniana stem extract and a Vanda coerulea stem extract, in proportions that can vary greatly.

In addition to the extract of the invention, said cosmetic or dermatological composition comprises at least one cosmetically or dermatologically acceptable excipient which can be chosen from pigments, dyes, polymers, surfactants, rheology agents, fragrances, electrolytes, pH modifiers, anti-oxidants and preservatives, and mixtures thereof.

The cosmetic or dermatological composition according to the invention may, for example, be a serum, a lotion, an emulsion, a cream, in particular a tinted cream, or else a hydrogel, preferably a mask, or may be in the form of a stick or else a patch.

The compositions of the invention have an effect that is particularly desired for reducing or delaying the effects of skin ageing, and make it possible in particular to obtain a protective, corrective, restructuring effect when they are applied to the skin of the face or of the body.

The present invention relates, as previously disclosed, to a use of the extracts as defined above as a cosmetic or dermatological agent for maintaining the structure of the skin, in particular by limiting the degradation of the extracellular matrix of the upper layers of the dermis and of the epidermis, and/or for reducing or delaying the effects of skin ageing, in particular the formation of wrinkles, and/or for obtaining a protective, corrective or restructuring effect on the skin, and/or as an anti-inflammatory agent.

As clearly emerges from the examples hereinafter, the applicant has demonstrated the advantages of the extracts of the plant Vanda teres, optionally combined, in addition, with other Vanda extracts for improving the activities listed below:
anti-inflammatory activity through inhibition of the secretion of inflammation mediators IL-8 and PGE₂,
free-radical-scavenging properties, preserving the skin against damage caused by oxidative stress,
synthesis of fibronectin fibers and collagen type I fibers, which are fibers of the extracellular matrix for maintaining the structure of the skin,
inhibition of the MMP-2 and 9 enzymes which degrade the fibers of the extracellular matrix.

Thus, the invention also relates to the use of the Vanda teres extracts defined above, as a cosmetic or dermatological agent or for the production of a cosmetic composition for maintaining the structure of the skin, in particular by limiting the degradation of the extracellular protein matrix of the upper layers of the dermis and of the epidermis, and thus reducing or delaying the effects of skin ageing, in particular the formation of wrinkles, or else for obtaining a protective, corrective or restructuring effect.

A subject of the invention is also a cosmetic care method using the cosmetic compositions comprising an effective amount of said extract, for contributing to maintaining the integrity of the structure of the skin, in particular by stimulating fibronectin synthesis, by inhibiting the MMP-2 and 9 enzymes responsible for collagen fiber degradation and by increasing the expression of the gene encoding interleukin IL-10.

Said method of cosmetic care comprises the application of a composition as described above and comprising an extract as defined above, to at least one area, of the skin of the face or of the body, that is concerned.

In the examples, all the percentages are given by weight, the temperature is in degrees Celsius, and the pressure is atmospheric pressure, unless otherwise indicated.

In addition to the orchids of the Vanda genus, the plant material from which the extract of the invention is prepared may comprise one or more extracts of other plants, in the form of extracts of whole plants or of parts of plants.

These plants may be either orchids of a genus other than the Vanda genus, or plants of another family, known to have properties similar or complementary to those demonstrated for orchids of the Vanda genus.

Plants of which the extracts are known to slow down or prevent the effects of skin ageing, by various mechanisms such as maintaining the integrity of the structure of the skin or an action on wrinkles, may in particular be chosen.

Other purposes, characteristics and advantages of the invention will become clearly apparent in the light of the explanatory description which follows, given with reference to the examples of preparation of extracts and to examples of cosmetic compositions using such extracts, given simply by way of illustration and which cannot therefore in any way limit the scope of the invention.

### EXAMPLES

### I. Preparation of Extracts

### Example 1

### Preparation of a Vanda teres Stem Extract (Extract n° 1 According to the Invention)

The plant material (plant stems) in the dry state is ground extemporaneously.

10 g of ground plant are introduced into a 250 mL round-bottomed flask, into which 150 mL of an ethanol/water mixture (90/10 v/v) are introduced.

The round-bottomed flask surmounted with a ball condenser with magnetic stirring is placed in a thermostatic bath, and then heated to the reflux of the solvent.

The reflux is maintained for 30 minutes with stirring.

Once the heating has been stopped, the round-bottomed flask is left to cool to ambient temperature outside the bath.

The mixture is then vacuum-filtered through a Buchner funnel with a Whatman 70 μm GF/F filter and a tared flask: filtrate 1 is obtained.

The cake is washed on the Buchner funnel with 50 mL of the extraction solvent: filtrate 2 is obtained.

The two filtrates are combined and weighed.

The filtrate thus obtained is introduced into a pre-tared round-bottomed flask, and then concentrated to dryness on a rotary evaporator in a water bath placed at a maximum temperature of 50° C.

A dry residue is obtained, which is quantified in order to determine the extraction yield by mass, expressed relative to the mass of dry plant introduced.

The extraction yield expressed as mass of dry extract obtained per 100 g of starting plant material in the dry ground state is 12.4.

### Example 2

### Preparation of a Vanda Teres Root Extract (Extract n° 2 According to the Invention)

The same protocol to that used to prepare extract n° 1 of example 1 is used, but it is applied to a plant material constituted of Vanda teres roots.

An extract, hereinafter denoted extract n° 2 of the invention, is obtained with an extraction yield of 14.8.

### Example 3

### Preparation of Other Extracts of Parts of a Plant of the Vanda Genus

The same extraction protocol as that defined in example 1 is used to prepare various extracts of Vanda teres and Vanda coerulea.
Vanda denisoniana stem extract (extraction yield of 10.6);
Vanda coerulea stem extract (extraction yield of 8.9);
Vanda coerulea root extract.

These various extracts will be used in the examples which follow, either separately, or as a mixture and combined in various proportions with the extract of example 1 and/or with the extract of example 2.

### II. Protocol and Materials and Methods for the Activity Tests

The doses for using the extracts are predetermined by means of the XTT test (reagent: Tetrazolium salt) on a cell culture of two cell lines, HaCaT cells (line of keratinocytes immortalized in vitro) and normal human fibroblasts (NHFs).

1. Inflammation Markers

1-1) assay of interleukin-8 (IL-8)

Inflammation is a normal and immediate physiological response to any attack of mechanical or infectious nature.

Excessive production of oxygen free radicals (ROSs) leads to the release of certain cytokines responsible for the pro-inflammatory phenomenon, resulting in an acceleration of skin ageing.

An increase in interleukin-8 (IL-8) can then be observed in the epidermis: the keratinocytes participate actively in the immune defense by secreting cytokines, including IL-8, which creates a chemotactic gradient that attracts the cells involved in inflammation: T lymphocytes, neutrophils, etc. It has been demonstrated that keratinocytes express and release IL-8 when stimulated with interleukin-1β.
protocol

The HaCaT cells are seeded in a proportion of 10 000 cells/well (supplemented KSFM culture medium, Gibco) in a 96-well microplate. The plate is placed in an incubator (37° C. and 5% CO₂) for 24 hours.

The extracts tested, at the doses for use predetermined by means of the XTT test, are brought into contact with the cells after suitable dilution in the culture medium.

In parallel, the cells are stimulated with IL-18 at 25 ng/mL (R&D Systems).

One column is treated with cholecalciferol at 0.39 μg/mL (Sigma) and stimulated (in order to obtain the positive control), and one column is nontreated and stimulated with IL-1β.

The nontreated cell column is brought into contact with the solubilization solvent DMSO (control condition).

Each concentration of product, the positive control and the treatment-free control are evaluated on four wells of normal human keratinocytes (NHKs).

After 24 hours of treatment, the culture supernatants are sampled and stored at −20° C.

The assay is carried out according to the protocol described in the “Human IL-8/NAP-1 ELISA” kit (Abcys).

This test makes it possible to assay, by means of spectrophotometric measurement at 450 nm, the amount of IL-8 present in the supernatants, proportional to the absorbence measured.

1-2) Assay of Prostaglandins E2 (PGE2)

An increase in the level of PGE2 can occur under certain pathological conditions, such as inflammation or certain cancers.

PGE2 is the major mediator of inflammatory response phenomena during tissue damage.
protocol

The HaCaT cells are seeded in a proportion of 10 000 cells/well (supplemented KSFM culture medium, Gibco) in a 96-well microplate.

The plate is placed in an incubator (37° C. and 5% CO₂) for 24 hours.

The extracts tested are brought into contact with the cells, after suitable dilution in the culture medium.

One column is treated with indomethacin at 1 μg/mL (Sigma) in order to obtain a positive control, and one column is nontreated but brought into contact with DMSO.

Each concentration of product, the positive control and the treatment-free control are evaluated on four wells of NHKs.

After 24 hours of treatment, the culture supernatants are sampled and stored at −20° C.

The assay is carried out according to the protocol described in the PGE2 assay kit from R&D Systems.

This technique makes it possible to assay, by means of spectrophotometric measurement at 450 nm, the amount of PGE2 present in the supernatants, which is inversely proportional to the absorbence measured.

2. Measurement of the Expression of the Interleukin-10 (IL-10) Messenger

IL-10, described as a factor that inhibits the production of cytokines by Th1 lymphocytes and the effector functions of monocytes/macrophages, is in fact a cytokine capable of acting on many targets.

The main function thereof appears to be the inhibition of the inflammatory response.

IL-10 also regulates the proliferation and differentiation of certain inflammation cells or keratinocytes.
protocol

The effect of the orchid extracts is evaluated by means of the RT-QPCR method on human keratinocytes in monolayer (NHEKs) at normal confluence (molecular biology standard confluence), in SFM medium with a low calcium content and without supplements, in suitable formats.

The extracts and the control medium are tested for 24 hours, and then total RNA extraction is carried out, followed by DNAse treatments and a reverse transcription according to the standard protocols.

A quantitative PCR in triplicate made it possible to measure the expression of the IL-10 marker, related back to the G3PDH reference marker.

3. Markers for Maintenance of the Structure of the Skin

The tests are carried out on normal human fibroblasts (NHFs), which are dermal cells that promote maintenance of the structure of the skin.

The NHFs are seeded at the density of 5000 cells/well, and 200 μL/well of MEM culture medium (Gibco Invitrogen) supplemented with glutamine (Gibco Invitrogen, final concentration 2 mM) and 10% of FCS (foetal calf serum), in a 96-well microplate (Falcon).

The edges of the plate will not contain any cells, and the culture plate is placed in an incubator for 24 hours in order to obtain 80% cell confluence.

3-1) Assaying of the MMP-2 and 9 Enzymes

The function of the “Matrix metalloproteinase” (MMP) enzymes is to degrade the proteins of the extracellular matrix.

They play an important role in many normal physiological processes, such as embryonic development, morphogenesis and tissue remodeling.

MMP-2, also called gelatinase A, is expressed by the cells of the mesenchyme (mainly fibroblasts) during development and tissue regeneration. This expression is closely associated with cells of the neutrophil, macrophage and monocyte type.

With MMP-9, this protein degrades collagen type IV, a major compound of basal membranes and of gelatin (denatured collagen).

It can also degrade other types of collagens (V, VII and X), elastin and fibronectin.

MMP-9, also called gelatinase B, has three fibronectin type II domains and one domain homologous to collagen type V.

Pro-MMP-9 can be activated by MMP-3 or by certain bacterial proteinases.

The substrates for MMP-9 may be native collagens types IV, V, VII, X and XI, fibronectin, etc.
protocol

The extracts, at the doses for use predetermined by means of the XTT test, are brought into contact with the cells after suitable dilution in culture medium.

One column is treated with anogelline at 25 μg/mL, and one column is brought into contact with the solubilization solvent DMSO (control condition).

Each concentration of product, the positive control and the treatment-free control are evaluated on four wells of NHKs.

After 48 hours of treatment, the culture supernatants are sampled and stored at −20° C.

The assay for MMP-2 and 9 is carried out according to the protocols described in the Quantikine kits from R&D Systems, which make it possible to assay, respectively, by means of spectrophotometric measurement at 450 nm, the amounts of MMP-2 and MMP-9 present in the supernatants.

3-2) Assaying of Collagen I and Fibronectin Fibers
protocol

As previously, the extracts are brought into contact with the cells after suitable dilution in culture medium.

One column is treated with 0.1 μM vitamin C (ascorbic acid, Sigma) and one column is brought into contact with the solubilization solvent DMSO (control condition).

Each concentration of product, the positive control and the treatment-free control are evaluated on four wells of NHKs.

After 48 hours of treatment, the culture supernatants are sampled and stored at −20° C.

The assay is carried out according to the protocols described in the EIA kits from Takara.

These two tests use an immunoenzyme technique for assaying, respectively, by means of spectrophotometric measurement at 450 nm, the amounts of collagen and fibronectin fibers present in the supernatants.

4. Free-Radical-Scavenging Properties

The TBARs test, which measures the degree of protection of the membrane lipids of cells in culture, is carried out on the extracts.

The HaCaT cells are seeded, at 67 passages, in 24-well plates in a proportion of 75 000 cells and 1 mL per well. After incubation for 24 hours in an incubator (37° C., 5% CO₂) corresponding to 80% confluence, the cells are brought into contact with the orchid extracts.

A control constituted of plates of cells not treated with orchid extracts is also prepared.

The treatment is carried out in triplicate and at 1% (1 μL/mL of sample), for a period of 24 hours (37° C., 5% CO₂).

Two plates are processed in the same manner, for the treatment with the extract and for the control, in order to irradiate one of the two plates with UVA at a rate of 12 J/cm² (culture medium changed for PBS for the two plates).

At the end of the irradiation, the supernatants of the two plates are removed, and 500 μL of 0.8% SDS are added to each well of cells.

After five minutes of contact, the cells are recovered by scraping the wells.

250 μL of cells are brought into contact with 375 μL of 0.8% thiobarbituric acid and 375 μL of 20% acetic acid in stoppered pyrex tubes, brought to 95° C. for one hour in a water bath.

After a final centrifugation step, the supernatant is recovered in order to read the absorbence thereof at 532 nm.

The absorbence is measured for the sample subjected to the UV radiation and also for the nonirradiated sample, and is corrected in the following way:

Corrected absorbence=(absorbence measured−absorbence of water).

For the percentage lipoperoxidation inhibition, the calculation is carried out in the following way:

corrected absorbence (control)=(corrected absorbence (UV solvent)−corrected absorbence (solvent)).

The percentage inhibition for each sample is obtained by virtue of the formula: (corrected absorbence (UV sample)−corrected absorbence (nontreated sample))/corrected absorbence (control).

### Iii. Activities of the Extracts of the Invention

### 1. Anti-Inflammatory Activity

### Inflammation (HaCat)−assaying of IL-8 and of PGE₂

The extracts are tested at doses for use predetermined by means of the XTT test, which are respectively:
25 μg/mL for the plant roots,
50 μg for the plant stems,
12.5 μg for the leaves.

The percentages of inhibition of IL-8 and PGE₂ release are calculated for each treatment with an orchid extract, relative to the basal amount of IL-8 present in the nontreated culture supernatants.

The results are mentioned in the table below:

| Genus | Species | Organ | IL8 | PGE2 |
|---|---|---|---|---|
| Vanda | teres | Stems | −30% | −33% |
| Vanda | teres | Leaves | −14% | −67% |
| Vanda | teres | Roots | −8% | −57% |

A significant decrease in the release of the IL-8 and PGE₂ markers is observed.

This effect of inhibition on the release of these inflammation markers reflects an anti-inflammatory activity of the extract tested.

This anti-inflammatory activity blocks the pro-inflammatory phenomenon caused by the excess production of free radicals and thus prevents the acceleration of skin ageing.

2. Free-Radical-Scavenging Tests

The test carried out on a Vanda teres stem extract indicates a 41% inhibition of peroxidation.

3. Assaying of Procollagen Type I, of Fibronectin and MMP-2 and 9 Activity (NHFs)

The extracts are tested at the dose of 25 μg/mL.

The results are given in the table below:

| Genus | Species | Organ | Collagen | Fibronectin | MMP2 | MMP9 |
|---|---|---|---|---|---|---|
| Vanda | teres | Stems | 8% | 28% | −45% | −36% |
| Vanda | teres | Leaves | 27% | 0 | −34% | −15% |
| Vanda | teres | Roots | −20% | 0 | −43% | −10% |

The treatment of the cells with each of the extracts tested results in a considerable decrease in the synthesis of the metalloproteinase enzymes responsible for degradation of the extracellular matrix made in particular from collagen.

The extracts exhibit an inhibitory activity with respect to metalloproteinases, resulting in a cosmetic effect of prevention of skin ageing by slowing down of extracellular matrix degradation.

It will also be noted that the stem extract showed a significant action on the synthesis of collagen and of fibronectin and that the leaf extracts showed a particularly significant activity for collagen synthesis.

4. Expression of the Gene Encoding IL-10 (NHKs)

A Vanda teres stem extract results in a significant increase of 1000% in the expression of this gene compared with expression in a nontreated control.

Test Conclusions

It emerges from the above tests that the stem extracts exhibit an excellent potential in terms of overall biological activity of prevention or slowing down of the appearance of skin ageing signs, given the results obtained on the various markers.

### Iv. Biological Activity of Mixtures of Extracts of the Vanda Genus

In this example, a systematic study of the activity of prevention or of slowing down of the appearance of skin ageing signs and of the anti-inflammatory activity of a large number of mixtures of the following four types of extracts was carried out:
Vanda teres root extract,
Vanda denisoniana stem extract,
Vanda coerulea stem extract,
Vanda teres stem extract.

The various tests were carried out with a total concentration of extracts of 25 μg/mL for each of the tests, so as to be able to compare the effectiveness of the various mixtures with the effectivenesses obtained with each of the types of extracts taken alone.

In order to carry out this study, two biological models were more particularly retained:
expression of MMP-2 in NHF culture supernatants,
expression of PGE2 in HaCaT culture supernatants.

Each mixture or extract tested is prepared in such a way as to obtain a concentration corresponding to 25 μg/mL.

For each experiment, the values for the positive control and the values for the extracts are compared using the Student's test. Their difference is said to be significant when the value is less than or equal to 0.05.

The tests carried out made it possible to characterize a certain number of particularly advantageous mixtures exhibiting significant activities with respect to each of the two models studied, or even real synergy effects.

By way of advantageous mixtures, mention will be made of a mixture containing a Vanda teres root extract, a Vanda teres stem extract and a Vanda denisoniana stem extract in equivalent proportions (⅓ of each extract), and also a mixture of a Vanda teres root extract and of a Vanda denisoniana stem extract in equivalent proportions (50/50).

Mention will also be made of a mixture containing the four extracts mentioned above, in equivalent proportions (25%) and also a mixture having the following composition:
Vanda teres roots: 12.5%,
Vanda denisoniana stems: 62.5%,
Vanda coerulea stems: 12.5%,
Vanda teres stems: 12.5%.

All these mixtures prove to be particularly advantageous on each of the two models.

### V. Cosmetic Compositions of the Invention

### Example 4

### Cosmetic Composition Comprising the Extract of the Invention

The orchid extract used as active agent in the cosmetic composition of this example is obtained by reproducing the method of example 1, but applying it to a mixture of stems of Vanda denisoniana, of Vanda teres and of Vanda coerulea, in the dry and ground state and according to a 1/1/1 ratio of these three plants.

The dry extract thus obtained is then dissolved at 1% w/w in a 60/40 v/v mixture of glycerol/water.

This solution is used as active agent for preparing the following cosmetic composition (% expressed as w/w):

| Phase A | |
|---|---|
| Solution containing 1% of orchid extract | 0.3% |
| Phenoxyethanol | 0.5% |
| Xanthan gum | 0.2% |
| Acrylates/C20-30 alkyl acrylate crosspolymers | 0.15% |
| Tetrasodium EDTA | 0.1% |
| Water | qs |
| Phase B | |
| Hydrogenated polyisobutene | 4% |
| Squalane | 3% |
| Caprylic/capric triglyceride | 3% |
| Pentylene glycol | 3% |
| Glyceryl stearate | 3% |
| PEG-100 stearate | 2.5% |
| Beeswax | 1.5% |
| Dicaprylyl carbonate | 1.5% |
| Cetyl alcohol | 1% |
| Stearyl alcohol | 1% |
| Dimethicone | 1% |
| Phase C | |
| Sodium hydroxide | 0.04% |
| Water | qs 100% |

The gelling agents of phase A are dispersed in water and then the mixture is heated at 80-85° C., before solubilizing all the other compounds, including the solution of orchid extract.

The compounds of phase B are heated at 85° C. so as to form a homogeneous phase.

Phase A is emulsified in phase B using an Ystral mixer.

The oil/water emulsion thus obtained is finally neutralized with a % w/w aqueous sodium hydroxide solution, and then cooled.

The composition obtained is a cream intended to be applied to the face or a part of the face.

1. A cosmetic or dermatological composition, containing at least one extract of at least a part of an orchid of the species Vanda teres, in solution or as a dispersion in a cosmetically or dermatologically acceptable carrier compatible with topical application to the skin.

2. The composition as claimed in claim 1, wherein said extract is obtained from a part of the plant chosen from the group consisting of: the stem, the roots, the leaves of the orchid Vanda teres, and any mixture thereof.

3. The composition as claimed in claim 1, wherein said extract is a stem extract.

4. The composition as claimed in claim 1, wherein said extract is obtained by bringing at least a part of said orchid, into contact with at least one polar solvent.

5. The composition as claimed in claim 4, wherein said polar solvent is chosen from the group consisting of:
water, a C₁-C₄ alcohol, and a C₂ to C₆ glycol, and any mixture thereof.

6. The composition as claimed in claim 5, wherein said solvent is an aqueous-ethanolic mixture.

7. The composition as claimed in claim 4, wherein said polar solvent is a solvent in subcritical state.

8. The composition as claimed in claim 1, wherein said extract has been subjected to at least one decoloring and/or purifying step.

9. The composition as claimed in claim 1, wherein said extract is introduced into said composition after having been absorbed onto a support, in particular onto a support.

10. The composition as claimed in claim 1, wherein said extract is an aqueous extract.

11. The composition as claimed in claim 1, wherein the composition contains from 0.001% to 5%.

12. The composition as claimed in claim 1, wherein the composition further contains at least one extract of at least one other plant belonging to the orchid family (Orchidaceae).

13. The composition as claimed in claim 1, wherein the composition further contains at least one extract of at least one other orchid of the Vanda genus.

14. The composition as claimed in claim 13, wherein the composition contains at least one extract chosen from the group consisting of: a Vanda teres stem extract, a Vanda teres root extract, and any mixture thereof, in combination with a Vanda denisoniana stem extract, a Vanda coerulea stem extract, and any mixture thereof.

15. The composition as claimed in claim 1, wherein the composition comprises a Vanda teres root extract.

16. The composition as claimed in claim 1, wherein the composition is in the form of a serum, a lotion, an emulsion, a cream, a hydrogel, a mask, a stick or a patch film.

17. A method of cosmetic care for the skin intended for one or more care chosen from the group consisting of: maintaining the structure of the skin by limiting the degradation of the extracellular matrix of the upper layers of the dermis and of the epidermis, for reducing or delaying the effects of skin ageing and, the formation of wrinkles, for obtaining a protective, corrective or restructuring effect on the skin, and acting as an anti-inflammatory agent wherein the method comprises applying, to at least part of the body or of the face, of an extract as defined in claim 1, in a cosmetic or dermatological composition.

18. A method of cosmetic care for the skin, intended for obtaining an effect of prevention or slowing down of the appearance of skin ageing signs, wherein the method comprises applying, to at least a part of the body or of the face, of a cosmetic composition as defined in claim 1.

19. The composition as claimed in claim 1, further containing at least one extract chosen from the group consisting of an extract of at least a part of an orchid of the species Vanda denisoniana, an extract of at least a part of an orchid of the species Vanda coerulea, and any mixture thereof.
